(19) United States
(12) Patent Application Publication
(10) Pub. No.: US 2009/0286251 A1
(43) Pub. Date: Nov. 19, 2009

(54) Enzyme Reagents for Amplification of Polynucleotides in the Presence of Inhibitors

(75) Inventor: Yan Xu, Hamilton, MA (US)

Correspondence Address: HARRIET M. STRIMPEL, D. Phil., New England Biolabs, Inc., 240 COUNTY ROAD, IPSWICH, MA, 01938-2723, US

(73) Assignee: NEW ENGLAND BIOLABS, INC., Ipswich, MA (US)

(21) Appl. No.: 12/466,856

(22) Filed: May 15, 2009

Related U.S. Application Data
(60) Provisional application No. 61/053,740, filed on May 16, 2008.

(51) Int. Cl. C12Q 1/68; C12P 19/34; C12N 9/00

(52) U.S. Cl. 435/6; 435/91.2; 435/183

(57) ABSTRACT

Compositions and methods are provided for amplifying polynucletoides from samples containing inhibitors that normally inhibit amplification using an enzyme blend containing a plurality of polymerases. The ability to amplify polynucleotides efficiently in the presence of inhibitors allows the enzyme reagent to be used in both routine amplification and real-time amplification from inhibitor-containing samples.

### CROSS REFERENCE

This application claims priority from U.S. provisional application Ser. No. 61/053,740 filed May 16, 2008.

### BACKGROUND

Because of its sensitivity and robustness, amplification of nucleic acids by polymerase chain reaction (PCR) has been widely used in basic biological research, clinical research, and forensic studies. For most PCR amplification, DNA templates are first purified from biological samples because of prevalent contaminants or inhibitors in the raw materials such as blood, soil, and tissue. Substantial reductions in PCR amplification yields have been noted in the presence of inhibitors that occur in biological samples (Al-Soud and Radstorm, J. Clin. Microbiol. 38:4463-4470 (2000)). Although common purification procedures can remove some PCR inhibitors to a certain degree and allow successful PCR amplification, the additional pre-treatment steps are undesirable. First, it is time-consuming to perform DNA purification from a large number of samples. Second, contaminant DNA may be introduced during preparation. Third, DNA purification may cause uneven DNA recovery, leading to false negative results or unreliable DNA quantification by PCR (Kramvis et al. J. Clin. Microbiol. 34: 2731-2733 (1996)).

One common biological sample is whole blood, which is used for diagnosis of genetic diseases, viral/bacterial infections, and blood typing. However, PCR analysis of blood samples is hindered by PCR-inhibitory compounds present in blood samples. A few known inhibitors are heme, iron, porphyrins, hemoglobin, immunoglobulin G, bile, lactoferrin, proteases, and anticoagulants (Al-Soud and Radstrom, J Clinical Microbiol 39:485-493 (2001); Kreader, Applied and Environmental Microbiology 62:1102-1106 (1996); and Akane, J. Forensic Sciences 39:362-372 (1994)). Mechanisms of inhibition can be one of the followings: direct inhibition of polymerase, chelation of magnesium, and binding of template DNA (Akane, J. Forensic Sciences 39:362-372 (1994); Al-Soud and Radstrom, J Clinical Microbiol. 39:485-493 (2001); and Sefers et al. Reviews in Medical Microbiology 16:59-67 (2005)). It is reported that as little as 0.2% whole blood can inhibit PCR by Taq DNA polymerase (Al-Soud and Radstrom, J Clinical Microbiol. 38:4463-4470 (2000)).

Different protocols have been developed to remove inhibitors from blood. One purification method involves proteinase K treatment followed by phenol extraction and DNA precipitation (Ahmad, et al. J Med Genet. 32(2):129-130 (1995)). Another simpler sample processing method involves alkaline release of DNA and neutralization before an aliquot can be used for PCR (Rudbeck and Dissing, Biotechniques 25:588-592 (1998)). Commercial DNA purification kits are also developed for purification of DNA from blood (Rabodonirina et al. J Clinical Microbiol. 37:127-131 (1999); and Angelini et al. Pathophysiol Haemos Thromb 32:180-183 (2002)).

It is desirable to carry out PCR amplification directly from blood samples. One approach is to optimize buffer components to enhance polymerase activities in the presence of blood inhibitors. Both BSA and detergents have been shown to increase blood tolerance by Taq DNA polymerase up to about 2% whole blood (Al-Soud and Radstrom, J Clinical Microbiol. 38:4463-4470 (2000), Bu et al. (Anal Biochem. 375:370-372 (2008)).

Another approach is to develop thermostable DNA polymerases resistant to the inhibitors present in blood. Some mutant Taq DNA polymerases have also been shown to be able to amplify specific DNA sequences in the presence of up to 20% blood (PCT Publication No. WO2005/113829). Phusion® Flash Master Mix can tolerate up to 20% whole blood in a PCR reaction (Finnzymes, Espoo, Finland).

### SUMMARY OF THE INVENTION

Embodiments of this invention relate to a method of using polymerase mixtures containing a plurality of DNA polymerases including a Family A DNA polymerase and a Family B exo⁻ DNA polymerase for amplifying polynucleotides in the presence of inhibitors such as blood, SYBR® (Invitrogen, Carlsbad, Calif.), humic acid and detergents. The ability to amplify polynucleotides efficiently in the presence of inhibitors allows the enzyme reagent to be used for inhibitor-containing samples in both routine amplification and real-time amplification.

In an embodiment of the invention, a method is provided that includes adding to a preparation containing a polynucleotide and at least one amplification inhibitor, a mixture containing a Family A DNA polymerase and a Family B exo⁻ DNA polymerase in a buffer. The combination of a plurality of polymerases is referred to herein as a blend. This mixture is capable of enhancing polynucleotide amplification including real time PCR synergistically. Enhanced yields of amplified target DNA using both Family A and Family B exo⁻ DNA polymerases were detected using gel electrophoresis as compared with the yields of amplified DNA obtained using only a Family A DNA polymerase or a Family B exo⁻ DNA polymerase.

In embodiments of the invention, a Family A DNA polymerase used in the above method may include one or more of the following: Taq DNA polymerase, Tbr DNA polymerase, Tth DNA polymerase, Tfl DNA polymerase, Tfil DNA polymerase, Tru DNA polymerase and Rob DNA polymerase.

In embodiments of the invention, a Family B exo⁻ polymerase of the above method may include one or more of the following: Vent® exo- DNA polymerase (New England Biolabs, Inc. (NEB), Ipswich, Mass.), Deep Vent™ exo⁻ DNA polymerase (NEB, Ipswich, Mass.), 9° N exo⁻ DNA polymerase (NEB, Ipswich, Mass.), Pfu exo⁻ DNA polymerase, Pwo exo⁻ DNA polymerase, KOD exo⁻ DNA polymerase, Tgo exo- DNA polymerase, JDF-3 exo- DNA polymerase, and Tma exo- DNA polymerase. Where the amplification method is PCR, it is preferable that the Family A and B exo⁻ DNA polymerases be thermostable.

In embodiments of the invention, examples of the at least one inhibitor referred to herein includes: whole blood, blood components, anticoagulants, SYBR® green I (Invitrogen, Carlsbad, Calif.), humic acid, and detergents such as SDS. For example, a preparation of a polynucleotide may contain whole blood such that the preparation and the mixture taken together contain the whole blood at a concentration in the range of at least 0.01% to at least 40% (blood volume/total preparation volume). Whole blood as a liquid or dry blood stored on a paper such as a Guthrie card or FTA paper may be added to the mixture for amplification of target DNA.

Embodiments of the method can be used for quantifying specific target DNA from biological samples such as blood or feces, or environmental samples such as soil. Quantitative detection of the target DNA can be achieved using dyes or fluorogenic compounds such as SYBR® green I (Invitrogen, Carlsbad, Calif.) or Eva green (Biotium, Hayward, Calif.). A predetermined concentration of SYBR green I for example, at least about 1× to 80×, may be used for this purpose. Additionally or alternatively, amplification may be detected using hybridization probes such as hydrolysis probes (for review see Valasek and Repa, Adv Physiol Educ 29:151-159 (2005)) and molecular beacons (for review see Tyagi and Kramer, Nature Biotechnology 14:303-308 (1995)). Hydrolysis probes are also called 5′ nuclease probes, including the most commonly used TaqMan® probe (Applied Biosystems, Foster City, Calif.). Hydrolysis probes are sequence-specific dually fluorophore-labeled DNA oligonucleotides with one fluorophore label at one end and a fluorescence quencher at the other end. Both labels are in close proximity so that the fluorescence is quenched unless the fluorophore is released by the 5′-3′ nuclease activity of the polymerase. Finally, amplification may also be detected using labeled primers such as LUX primer (Rekhviashvili, Molecular Biotechnology 32(2):101-110(10)(2006)).

In an embodiment of the invention, an enzyme blend is provided that includes a Family A DNA polymerase and a Family B exo- DNA polymerase. The enzyme blend is capable of amplifying a polynucleotide in the presence of an inhibitor such as found in a biological sample, for example, blood or fecal matter, or an environmental sample such as soil, or SYBR® green I (Invitrogen, Carlsbad, Calif.), or a detergent. In an additional embodiment, the DNA polymerases in the enzyme blend are thermostable.

### BRIEF DESCRIPTION OF THE DRAWINGS

FIG. 1 shows the results of amplifying a 2.0 kb or a 2.9 kb target DNA in the presence of 10% blood (blood volume/total reaction volume) using Taq DNA polymerase and Vent® exo- DNA polymerase separately and together as indicated. Unit concentrations are shown in the figure. The final PCR reaction contained 60 mM Tricine pH 8.7, 3 mM MgCl₂, 0.2 mM EGTA, 0.3 mM dNTPs, and 300 nM of each of the primers SEQ ID NOS:1 and 2 or SEQ ID NOS:3 and 4. These were used to amplify a 2.0 kb fragment of Dnmt1 and a 2.9 kb of Il-7Ra gene, respectively. The PCR conditions were 95° C. for 2 min followed by 30 cycles of 95° C., 20 sec, 57° C., 30 sec, and 68° C., 4 mins.

A synergistic effect was observed in lanes 1, 4, 7, 10, 13 and 16 when the two polymerases were used together compared to their use separately in lanes 2, 3, 5, 6, 8, 9, 11, 12, 14, 15, 17 and 18.

FIG. 2 shows amplification of a 2.0 kb target DNA (Dnmt1) from 5% human blood using Taq DNA polymerase and/or Deep Vent™ exo⁻ DNA polymerase in the amounts shown above each lane. The final PCR reaction (25 μl) contained 60 mM Tricine pH 8.7, 3.5 mM MgCl₂, 5 mM (NH₄)₂SO₄, 6% glycerol, 0.3 mM dNTPs, and 300 nM of each primer SEQ ID NOS:1 and 2. PCR conditions were 95° C. for 2 min followed by 32 cycles of 95° C., 30 sec, 60 ° C., 30 sec, and 68 ° C., 3 min. Lanes 1, 4 and 7 show enhanced yield after amplification using a polymerase blend.

FIG. 3 shows amplification of a 0.68 kb target DNA (CC-chemokine receptor 5 (CCR5)) in 0%-40% human blood using an enzyme blend of 2 units Taq DNA polymerase and 2 units of Vent® exo⁻ polymerase. The final PCR reaction (50 μl) contained 60 mM Tricine pH 8.7, 3.5 mM MgCl₂, 5 mM (NH₄)₂SO₄, 60% glycerol, 0.3 mM dNTPs, and 300 nM of each primer SEQ ID NOS:5 and 6. The sample of blood containing the target DNA to total reaction volume (vol/vol) is shown above each lane. PCR amplification conditions were 95° C. for 5 min followed by 40 cycles of 95° C., 30 sec, 60° C., 30 sec, and 68° C., 3 min. Amplification products could be detected even at 40% blood in the reaction mixture.

FIG. 4 shows amplification of a 1.1 kb DNA (Dnmt1) in 4% human blood containing various anticoagulants using an enzyme blend of Taq DNA polymerase (5 units) and Vent® exo- DNA polymerase (2 units). The final PCR reaction (50 μl) contained 60 mM Tricine pH 8.7, 3.5 mM MgCl₂, 5 mM (NH₄)₂SO₄, 60% glycerol, 0.3 mM dNTPs, and 300 nM of each primer SEQ ID NOS:1 and 7. The final reaction volume was 50 μl. PCR conditions were 95° C. for 2 min followed by 35 cycles of 95° C. 30 sec, 60° C. 30 sec, and 68° C. 2 min. Blood samples were purchased from Innovative Research Inc., Novi, Mich.
Lane 1: Blood containing potassium EDTA at approx 0.85 grams per 450 ml blood;
Lane 2: Blood containing sodium EDTA at approx 0.85 grams per 450 ml blood;
Lane 3: Blood containing sodium citrate at approx 0.105 M;
Lane 4: Blood containing sodium heparin at approx 10,000 units per 450 ml blood;
Lane M: 2-log DNA ladder (Catalog #N3200, NEB, Ipswich, Mass.).

FIG. 5 shows results of amplification of 2.8 kb DNA (Caspase 8) from 4% mouse whole blood and an enzyme blend of Taq DNA polymerase (5 units) and Vent® exo- (2 units) DNA polymerase.

The final PCR reaction (25 μl) contained 50 mM Tris-HCl pH 9.1, 3.5 mM MgCl₂, 16 mM (NH₄)₂SO₄, 0.1% Tween20, 0.3 mM dNTPs, and 300 nM of each primer: SEQ ID NOS:8 and 9 for the 1.2 kb Caspase 1 (lane 1); SEQ ID NOS:10 and 11 for the 2.8 kb Caspase 8 (lane 2); SEQ ID NOS:12 and 13 for the 4.0 kb Bcl2-like (lane 3), SEQ ID NOS:23 and 24 for the 0.34 kb Y chromosome (lane 4), and SEQ ID NOS:21 and 22 for the 0.2 kb Homer (lane 5). The final reaction volume was 25 μl. PCR conditions were 95° C. for 2 min followed by 35 cycles of 95° C., 30 sec, 60° C., 30 sec, and 68° C., 5 min.

The results show that the enzyme blend can amplify efficiently from 4% mouse whole blood.

FIG. 6 shows results of amplification of specific targets from dried whole blood on a Guthrie card (cat #10534612, Whatman) where a portion of a Guthrie card was placed into an amplification reaction mixture containing an enzyme blend of Taq DNA polymerase (2 units) and Vent® exo- (2 units) DNA polymerase. About 1 μl mouse blood was dotted on paper and air dried. A disk of 1 mm diameter containing the dry blood was punched out using a paper punch and dropped into a 25 μl reaction containing 60 mM Tricine pH8.7, 3.5 mM MgCl₂, 5 mM (NH₄)₂SO₄, 6% glycerol, 0.3 mM dNTPs, and 300 nM of each primer: SEQ ID NOS:14 and 15 for the 0.24 kb Zinc finger protein 198 (lane 1); SEQ ID NOS:8 and 9 for the 1.2 kb Caspase 1 gene (lane 2); and SEQ ID NOS:10 and 11 for the 2.8 kb Caspase 8 gene (lane 3). PCR conditions were 95° C. for 3 min followed by 35 cycles of 95° C. 30 sec, 60° C. 30 sec, and 68° C. 6 min.

FIG. 7 shows the results of amplifying a 1.1 kb DNA target from 4% human whole blood in the presence of SYBR® green I at 40×, 20×, 19×, 5×, 2.5×, 1×, and 0 using an enzyme blend of Taq DNA polymerase (5 units) and Vent® exo- polymerases (2 units) in 25 μl PCR reactions. The final PCR reaction contained 60 mM Tricine pH 8.7, 3.5 mM MgCl₂, 5 mM (NH₄)₂SO₄, 6% glycerol, 0.3 mM dNTPs, and 300 nM of each primer SEQ ID NOS:1 and 7. PCR condition was 95° C. for 2 min followed by 40 cycles of 95° C., 30 sec, 60° C., 30 sec, and 68° C. 2 min.

FIG. 8 shows the results from a real-time PCR amplification using an enzyme blend of Taq DNA polymerase (2.5 units) and Vent® exo⁻ DNA polymerase (2 units) to amplify a 2.1 kb DNA in a bacterial genome in 5% human whole blood and 12× SYBR® green I (Invitrogen, Carlsbad, Calif.). A series dilution of E. coli genomic DNA (10⁶, 10⁵, 10⁴, 10³, 10² copies) was analyzed in a BioRad iCycler™ qPCR machine. The final PCR reaction (25 μl) contained 60 mM Tris-sulfate pH 9.2, 3.5 mM MgCl₂, 20 mM (NH₄)₂SO₄, 5% glycerol, 0.3 mM dNTPs, 1 mM DTT, 0.06% NP40, 0.05% Tween20, 400 nM of each of seq16 and seq17 and the polymerase blend in a total 25 μl reaction. PCR conditions were 95° C. for 2 min followed by 45 cycles of 95° C. 30 sec, 60° C., 30 sec, and 68° C., 30 sec.

FIG. 9 shows the product of amplification from human genomic DNA in the presence of different amount of SDS (g/100 ml) as a percentage of the total reaction mixture. Taq DNA polymerase (2 units) and/or Vent® exo⁻ polymerase (2 units) were used. The final PCR reaction (25 μl) contained 60 mM Tricine pH 8.7, 3.5 mM MgCl₂, 5 mM (NH₄)₂SO₄, 6% glycerol, 0.3 mM dNTPs, and 300 nM of each primer: SEQ ID NOS:18 and 19 for the 2.1 kb DNA (Beta globin) (FIG. 9A) and SEQ ID NOS:18 and 20 for the 4.1 kb DNA (Beta globin) (FIG. 9B). PCR conditions were 95° C. for 30 sec followed by 35 cycles of 95° C. 30 sec, 60° C. 30 sec, and 68° C. 5 min.

FIG. 10 shows the results of amplification of DNA in mouse tissue using an enzyme blend of Taq DNA polymerase and Vent exo- DNA polymerase. About 1 m³ mouse tail tissue was added to 25 μl PCR reactions. The final PCR reaction contained 60 mM Tricine pH 8.7, 3.5 mM MgCl₂, 5 mM (NH₄)₂SO₄, 6% glycerol, 0.3 mM dNTPs, 5 units of Taq DNA polymerase, 2 units of Vent® exo- DNA polymerase and 400 nM of each primer SEQ ID NOS:8 and 9. PCR conditions were 95° C. for 3 min followed by 35 cycles of 95° C. 20 sec, 60° C. 30 sec, and 68° C. 1 min. M, NEB 2-log DNA ladder.

### DETAILED DESCRIPTION OF EMBODIMENTS

Embodiments of the invention include an amplification method such as PCR amplification of a target polynucleotide using a combination (blend) of at least two DNA polymerases to provide enhanced levels of amplified DNA. The enzyme blend allows both DNA amplification and real-time PCR analysis directly from whole blood samples. Furthermore, the same blend can effectively amplify DNA in the presence of inhibitors that would inhibit amplification of polynucleotides using a similar concentration of a single DNA polymerase only.

It was found that an enzyme mix of a Family A DNA polymerase and a Family B exo⁻ DNA polymerase can act synergistically in amplification of samples containing inhibitory components.

DNA polymerases have been grouped into different families according to sequence similarities (for review see Perler et al. Adv Protein Chem. 48:377-435 (1996)). Members of Family A polymerases include many bacterial and bacteriophage polymerases, which share significant similarity to Escherichia coli (E. coli) polymerase I; hence family A is also known as the pol I family. The Family A polymerases have a C-terminal polymerase domain and an N-terminal 5′-3′ exonuclease domain. Several pol I-like DNA polymerases have been cloned from hyperthermophilic eubacteria (organisms with an optimal growth temperature of at least 80° C. which also grow at 90° C., for review see Adams, Annual Review of Microbiology 47:627-658 (1993)), for example, Taq from Thermus aquaticus, Tth from Thermus thermophilus, Tfl from Thermus flavus, Tfil from Thermus filiformis, Tru from Thermus ruber, Tbr from Thermus brochianus, and Rob from Rhodothermus obamensis (Al-Soud and Radstrom (J. Clin. Microbiol. 38: 4463-4470 (2000)) showed that the Family A Taq DNA polymerase was completely inhibited by as little as 0.2% blood (vol/vol) in a standard buffer (10 mM Tris-HCl, 1.5 mM MgCl₂, 50 mM KCl, pH 8.3).

Family B exo⁻ DNA polymerases are pol alpha-like polymerases and include many eukaryotic DNA polymerases and archaeal DNA polymerases (for review, see Perler et al. Adv Protein Chem. 48:377-435 (1996); and Sousa, Trends Biochem. Sci. 21:186-190 (1996)).

Six regions of similarity (numbered from I to VI) are found in all or a subset of the Family B DNA polymerases. Most, if not all, sequences in the Family B DNA polymerases contain a characteristic DTDS motif. Amplification of polynucleotides can be achieved using a variety of methodologies that rely on DNA polymerases as described in the art. These amplification protocols may be isothermal or can be achieved using thermocycling. Polymerase chain reaction amplification is commonly used and is the subject of the examples. However, the methods described herein are applicable to other amplification methodologies.

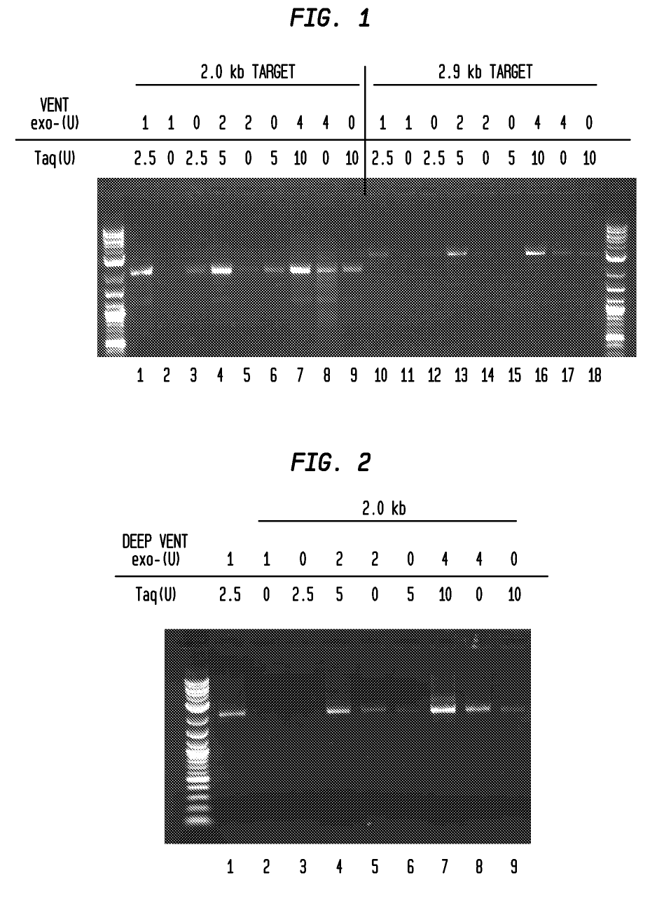

Family B exo⁻ DNA polymerases can be derived from DNA polymerases that naturally have 3′-5′ exonuclease activity by changing the conserved, critical residues in the 3′-5′ exonucleolytic domain as described in Bernad et al. Cell 59(1): 219-228 (1989); Derbyshire et al. Science 240(4849): 199-201 (1988); and U.S. Pat. Nos. 4,942,130 and 5,352,778. Examples of specific Family B DNA polymerases include Vent® DNA polymerase (NEB, Ipswich, Mass.) from Thermococcus litoralus, Deep Vent™ DNA polymerase (NEB, Ipswich, Mass.) from Pyrococcus strain GB-D, Pfu DNA polymerase from Pyrococcus furiosus (see for example U.S. Pat. No. 6,191,267), and 9° N DNA polymerase from Thermococcus sp. (strain 9° N-7). 9° N exo⁻ DNA polymerase (NEB, Ipswich, Mass.), Pwo DNA polymerase (Roche, Basel, Switzerland), KOD DNA polymerase (Novagen, Madison, Wis.), Tgo DNA polymerase (Roche, Basel, Switzerland), JDF-3 DNA polymerase (Stratagene, La Jolla, Calif.), and Tma DNA polymerase (Stratagene, La Jolla, Calif.). In general, archaeal DNA polymerases have 3′-5′ exonuclease activity but not 5′-3′ exonuclease activity. Under optimized conditions, Vent® exo- DNA polymerase can amplify directly from blood (FIG. 1, lanes 5, 8, 17).

Using a blend of DNA polymerases, polynucleotide amplification was successfully achieved in the presence of a variety of inhibitors. Amplification yields were optimized under selected reaction conditions. For example, in one embodiment, reaction conditions include: a buffer pH range of 7-10, more particularly a pH range of 8.5-9.5, more particularly, a pH range of 8.5-9.0; and magnesium concentrations in the buffer in the range of 1-5 mM, more particularly 2-4 mM and more particularly greater than 3 mM. Amplification in whole blood under various conditions is shown in FIGS. 1 through 8. Improvements in PCR efficiency are not limited to the above-specified reaction conditions. For example, additives such as glycerol and detergents in the buffer can further improve the PCR yield. The unit concentrations of DNA polymerases within a blend can be varied. For example, a Family A DNA polymerase may be represented in the blend in a range of 1-100 units for a 50 μl reaction volume and a Family B exo⁻ DNA polymerase may be represented in the blend within a range of 0.5-50 units also in the 50 μl reaction volume. The ratio can be optimized using the assays described herein.

Whereas the examples describe a blend of two DNA polymerases, this does not preclude the addition to the reaction mixture of additional polymerases without limit in number. In general as applied to PCR, thermostable polymerases are desirable. For isothermal amplification or amplification performed at lower temperatures than PCR, polymerases that are stable at those temperatures may be used.

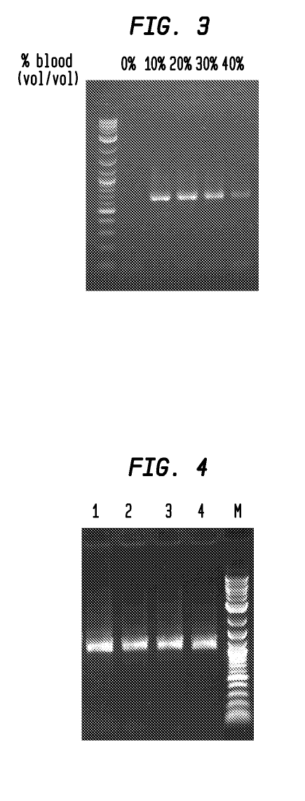

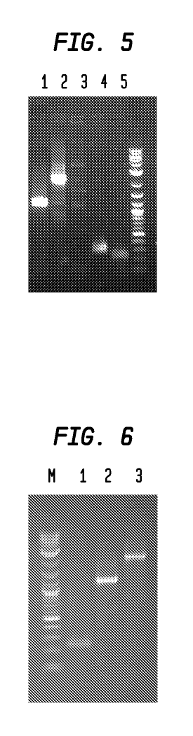

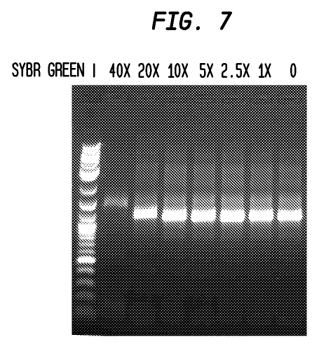

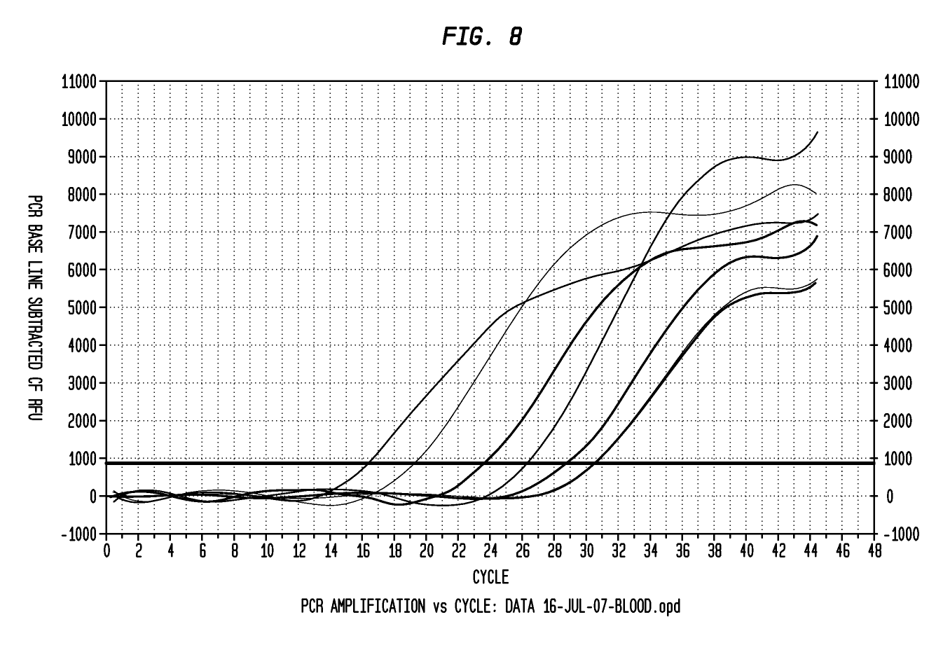

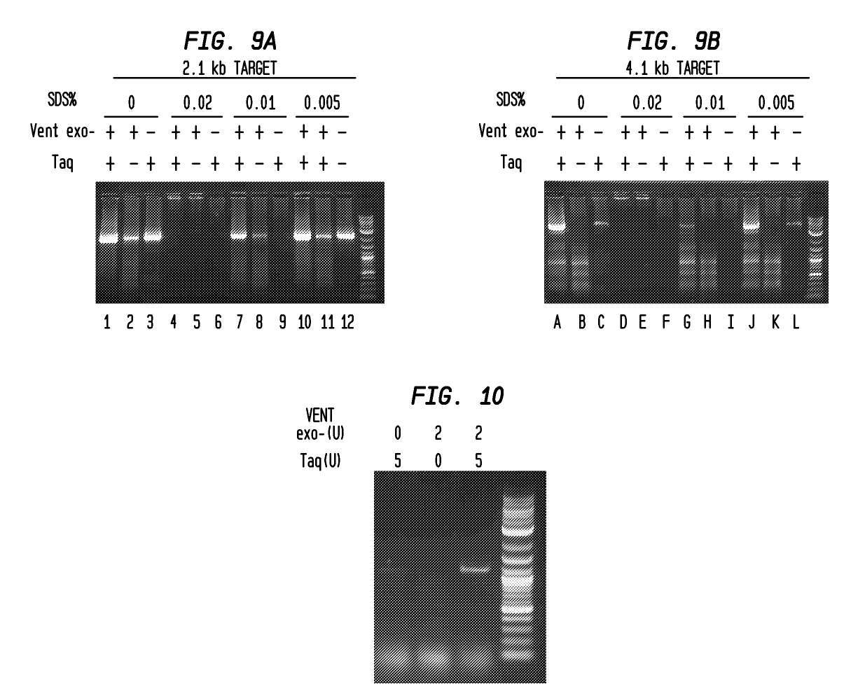

In the examples below, an enzyme mixture of Taq DNA polymerase and Vent® exo- DNA or Deep Vent™ exo⁻ polymerase showed synergistic effects on yield from PCR amplification of DNA in blood samples in a target size-independent manner (FIGS. 1, 2 and 5). A synergistic effect was also observed for blood dried onto paper (FIG. 6) and for tissue samples (FIG. 10). Embodiments describing enzyme mixtures showed that DNA targets from whole blood were amplified successfully even when the whole blood represented 40% of the reaction mixture (FIG. 3). The size of the target DNA for amplification by an enzyme blend is not limiting. In the examples, target DNA having a size of at least 4 Kb was found to be amplified in the presence of inhibitors (FIG. 5). The enzyme mixture was effective in producing enhanced yields of amplified DNA in the presence of a variety of inhibitors of PCR amplification including anticoagulants described in FIG. 5, SYBR® green I in FIG. 7 and SDS in FIG. 9.

The examples also show how an enzyme mixture of Taq DNA polymerase and Vent® exo- DNA polymerase can be used in real-time PCR detection from blood samples directly (FIG. 8). Furthermore, the 5′-3′ nuclease activity of Taq DNA polymerase allows the enzyme blend to be used with TaqMan®-based qPCR detection from blood samples directly.

### EXAMPLES

### Example I

### Direct amplification from Whole Blood Using Taq DNA Polymerase and Vent® exo⁻ DNA Polymerase

FIGS. 1 and 2 illustrate the advantageous effect of combining two polymerases into a blend for amplifying DNA in the presence of inhibitors. In FIG. 3, the enzyme blend of Taq DNA polymerase and Vent® exo⁻ DNA polymerase amplified a specific 0.68 kb fragment from whole blood where the whole blood was as much as 40% of the amplification reaction mixture. The blood-resistant property of the enzyme mix was tested with whole blood treated with four different anticoagulants: potassium EDTA, sodium EDTA, sodium citrate, and sodium heparin (FIG. 4).

The unit concentrations of the polymerases used herein can be varied and readily tested to observe the synergistic effect shown in the figures. Although the range of concentrations selected here showed a synergistic effect, it is anticipated that other enzyme unit concentrations could be used together to provide this observed synergy.

### Example II

### Direct Amplification from Mouse Whole Blood Using Tag DNA Polymerase and Vent® exo⁻ DNA Polymerases

Mice are commonly used as a model system for gene knockout studies. Screening for successful integration of foreign DNA into a specific genomic region is an important step in mouse genetic studies. A blood-direct PCR reagent can speed up the screening process by allowing PCR analysis at early stages from a single drop of blood without tedious genomic DNA purification. As shown in FIG. 5, amplicons of 0.2 kb-4.0 kb were successfully amplified from mouse whole blood using the enzyme blend of Taq DNA polymerase and Vent® exo⁻ DNA polymerase.

### Example III

### Direct Amplification from Mouse Whole Blood Stored on Paper

Clinical blood samples were either stored as liquid with anticoagulant present or as dry blood on paper. Amplification of three amplicons from mouse blood stored on a Guthrie paper was tested. A disk of 1 mm diameter was used in a 25 μl PCR reaction. As shown in FIG. 6, three specific bands were produced after 35 cycles.

### Example IV

### Direct Amplification from Whole Blood Using Enzyme Blend of Taq DNA Polymerase and Deep Vent™ Exo⁻ DNA Polymerase

To investigate whether the observed synergistic effect was a generalized effect between a thermostable Family A polymerase and Family B DNA polymerase, Vent® exo⁻ polymerase was replaced by Deep Vent™ exo⁻ DNA polymerase for amplifying specific fragments from blood directly. As shown in FIG. 2, Taq DNA polymerase and Deep Vent™ exo⁻ DNA polymerase also showed a synergistic effect (compare lane 1 with lanes 2 and 3; compare lane 4 with lanes 5 and 6; compare lane 7 with lanes 8 and 9). This illustrated that the synergistic effect can be generalized to a combination of any thermostable Family A DNA polymerase and Family B exo⁻ DNA polymerase.

### Example V

### Direct Amplification from Whole Blood in the Presence of SYBR®®Green I

Real-time PCR (qPCR) has been used in diagnostic studies. Real-time detection allows closed-tube analysis and provides quantitative data with minimal post-reaction handling. An enzyme blend of Taq DNA polymerase and Vent® exo⁻ DNA polymerase was used to amplify a specific DNA fragment from blood in the presence of up to 20× SYBR® green I (FIG. 7) demonstrating that the enzyme mix can be used in SYBR®-based qPCR detection.

In another experiment, a series dilution of E. coli genomic DNA with a range of 106, 105, 104, 103, 102 copies was detected in the presence of 5% human whole blood and 18× SYBR® green I (FIG. 8). A typical qPCR profile was obtained.

### Example VI

### Direct Amplification from Samples Containing SDS Using Tag DNA Polymerase and/or Vent® Exo⁻ DNA Polymerase

To investigate whether an enzyme mix of Taq DNA polymerase and Vent® exo⁻ DNA polymerase offered stronger amplification from samples containing other PCR inhibitors, 2 units of Taq DNA polymerase and/or 2 units of Vent® exo⁻ DNA polymerase were used to amplify a 2 kb or a 4 kb fragment in the presence of SDS. The enzyme mix of Taq DNA polymerase and Vent® exo⁻ DNA polymerase produced higher yield when used together than if enzymes were used separately (compare lane 7 with lanes 8 and 9; compare lane G with lanes H and I; compare lane J with lanes K and L).

TABLE 1

| Target | | | SEQ |
|---|---|---|---|
| DNA | Size | Primers | ID NO. |
| Dnmt1 | 2.0 kb | GGGGCACCTTCTCCAACTCAT | 1 |
| | | ACT | |
| | | CCTGAAACAAGGTTGTGGCAT | 2 |
| | | AGC | |
| Il-7Ra | 2.9 kb | CTCCAGAGATCAATAATAGCT | 3 |
| | | C | |
| | | TTGTCGCTCACGGTAAGTTCA | 4 |
| CCR5 | 0.68 kb | GCAGCGGCAGGACCAGCCCCA | 5 |
| | | AGATGACTATCT | |
| | | TGGAACAAGATGGATTATCAA | 6 |
| | | GTGTCAAGTCCA | |
| Dnmt1 | 1.1 kb | CCTCATTTGGGGAGGGGTTAT | 7 |
| | | CT | |
| Caspase 1 | 1.2 kb | CTGAAGGGTGGTGGTTCTGT | 8 |
| | | TCTTTCAAGCTTGGGCACTT | 9 |
| Caspase 8 | 2.8 kb | AACTGAACCCAGGTGGTCTG | 10 |
| | | TGTGGCAAAATGAGAGCAAG | 11 |
| Bcl2-like | 4.0 kb | GCAGGCTTTACACCCACAAT | 12 |
| | | AAACACGAGTTTGGGGTCAG | 13 |
| zinc | 0.24 kb | AGGTTCAGTCAGCCAGTGCT | 14 |
| finger | | | |
| protein | | | |
| 198 | | | |
| | | ACCAAAGCTTGATGCCAGTT | 15 |
| 16s RNA | 0.2 kb | AGCGGGGAGGAAGGGAGTAAA | 16 |
| | | GTT | |
| | | CAGTATCAGATGCAGTTCCCA | 17 |
| | | GGTT | |
| beta | 2.1 kb | ATCTTTCCAAACCCTCCCCGA | 18 |
| globin | | CAC | |
| | | CAGAACCCATAGAAACAAACC | 19 |
| | | GCACAC | |
| beta | 4.1 kb | GTTATTTAGGGGCTCTCCATA | 20 |
| globin | | CTGC | |
| homer | 0.2 kb | GCACCTATAAATTCCCAGCTT | 21 |
| | | GTCAG | |
| | | GGAAAAGCTACAGTAGGCACA | 22 |
| | | CAACC | |
| Y | 0.3 kb | CTGAAGCTTTTGGCTTTGAG | 23 |
| chromosome | | | |
| | | CCACTGCCAAATTCTTTGG | 24 |

What is claimed is:

1. A method, comprising:
adding to a preparation containing a polynucleotide and at least one amplification inhibitor, a mixture containing a Family A DNA polymerase and a Family B exo⁻ DNA polymerase in a buffer, such that the mixture is capable of enhanced amplification of the polynucleotide as determined by gel electrophoresis, compared with amplification in the presence of the Family A DNA polymerase or the Family B exo⁻ DNA polymerase only.

2. A method according to claim 1, wherein the amplification of the polynucleotide occurs by means of a polymerase chain reaction (PCR) and the Family A DNA polymerase and the Family B exo⁻ DNA polymerases are thermostable.

3. A method according to claim 2, wherein the Family A DNA polymerase is selected from the group consisting of: Taq DNA polymerase, Tbr DNA polymerase, Tth DNA polymerase, Tfl DNA polymerase, Tfil DNA polymerase, Tru DNA polymerase and Rob DNA polymerase.

4. A method according to claim 2, wherein the Family B exo⁻ DNA polymerase is selected from the group consisting of: Vent® exo⁻ DNA polymerase, Deep Vent™ exo⁻ DNA polymerase, 9° N exo⁻ DNA polymerase, Pfu exo⁻ DNA polymerase, Pwo exo⁻ DNA polymerase, KOD exo⁻ DNA polymerase, Tgo exo⁻ DNA polymerase, JDF-3 exo⁻ DNA polymerase, and Tma exo⁻ DNA polymerase.

5. A method according to claim 1, wherein the at least one inhibitor is selected from the group consisting of: whole blood, blood components, anticoagulants, SYBR® green I, humic acid, and SDS.

6. A method according to claim 1, wherein the preparation contains whole blood such that the preparation and the mixture contains the whole blood at a concentration in the range of at least 0.01% to at least 40% blood volume per total preparation volume.

7. A method according to claim 1, further comprising:
adding the whole blood to the preparation as a liquid or as dry blood stored on a paper such as a Guthrie card or FTA paper.

8. A method according to claim 1, wherein the PCR amplification is real time PCR (qPCR) and the Family A DNA polymerase is a thermostable polymerase and the Family B exo⁻ DNA polymerase is a thermostable polymerase.

9. A method according to claim 8, further comprising:
quantifying specific target sequences from biological samples or environmental samples using a predetermined concentration of SYBR® green I.

10. A method according to claim 9, wherein the biological samples are selected from blood or fecal samples or wherein the environmental sample is soil.

11. A method according to claim 9, wherein the SYBR® green I concentration is at least about 1× to 80×.

12. A method according to claim 8, further comprising:
detecting DNA amplification using hybridization probes.

13. A method according to claim 12, wherein the probe is a hydrolysis probe such as TaqMan® probe.

14. A method according to claim 12, wherein the probe is a molecular beacon.

15. A method according to claim 8, wherein fluorophore-labeled primers are used for quantitative detection.

16. An enzyme blend, comprising: a Family A DNA polymerase and a Family B exo⁻ DNA polymerase, the enzyme blend being capable of amplifying a polynucleotide in a biological sample.

17. The enzyme blend according to claim 16, wherein the biological sample is blood.

18. The enzyme blend according to claim 16, wherein amplifying the polynucleotide additionally occurs in the presence of inhibitors selected from the group consisting of: SYBR® green, a detergent and fecal matter.

19. The enzyme blend according to claim 16, wherein the DNA polymerases are thermostable.


SEQUENCE LISTING (24 sequences)

SEQ ID NO: 1 - DNA, 24 bp, artificial
  primer
ggggcaccttctccaactcatact

SEQ ID NO: 2 - DNA, 24 bp, artificial
  primer
cctgaaacaaggttgtggcatagc

SEQ ID NO: 3 - DNA, 22 bp, artificial
  primer
ctccagagatcaataatagctc

SEQ ID NO: 4 - DNA, 21 bp, artificial
  primer
ttgtcgctcacggtaagttca

SEQ ID NO: 5 - DNA, 33 bp, artificial
  primer
gcagcggcaggaccagccccaagatgactatct

SEQ ID NO: 6 - DNA, 33 bp, artificial
  primer
tggaacaagatggattatcaagtgtcaagtcca

SEQ ID NO: 7 - DNA, 23 bp, artificial
  primer
cctcatttggggaggggttatct

SEQ ID NO: 8 - DNA, 20 bp, artificial
  primer
ctgaagggtggtggttctgt

SEQ ID NO: 9 - DNA, 20 bp, artificial
  primer
tctttcaagcttgggcactt

SEQ ID NO: 10 - DNA, 20 bp, artificial
  primer
aactgaacccaggtggtctg

SEQ ID NO: 11 - DNA, 20 bp, artificial
  primer
tgtggcaaaatgagagcaag

SEQ ID NO: 12 - DNA, 20 bp, artificial
  primer
gcaggctttacacccacaat

SEQ ID NO: 13 - DNA, 20 bp, artificial
  primer
aaacacgagtttggggtcag

SEQ ID NO: 14 - DNA, 20 bp, artificial
  primer
aggttcagtcagccagtgct

SEQ ID NO: 15 - DNA, 20 bp, artificial
  primer
accaaagcttgatgccagtt

SEQ ID NO: 16 - DNA, 24 bp, artificial
  primer
agcggggaggaagggagtaaagtt

SEQ ID NO: 17 - DNA, 25 bp, artificial
  primer
cagtatcagatgcagttcccaggtt

SEQ ID NO: 18 - DNA, 24 bp, artificial
  primer
atctttccaaaccctccccgacac

SEQ ID NO: 19 - DNA, 27 bp, artificial
  primer
cagaacccatagaaacaaaccgcacac

SEQ ID NO: 20 - DNA, 25 bp, artificial
  primer
gttatttaggggctctccatactgc

SEQ ID NO: 21 - DNA, 26 bp, artificial
  primer
gcacctataaattcccagcttgtcag

SEQ ID NO: 22 - DNA, 26 bp, artificial
  primer
ggaaaagctacagtaggcacacaacc

SEQ ID NO: 23 - DNA, 20 bp, artificial
  primer
ctgaagcttttggctttgag

SEQ ID NO: 24 - DNA, 19 bp, artificial
  primer
ccactgccaaattctttgg